(19) United States
(12) Patent Application Publication
(10) Pub. No.: US 2021/0090747 A1
(43) Pub. Date: Mar. 25, 2021

(54) SYSTEMS AND METHODS FOR MODEL-ASSISTED EVENT PREDICTION

(71) Applicant: Flatiron Health, Inc., New York, NY (US)

(72) Inventors: Benjamin E. BIRNBAUM, Brooklyn, NY (US); Joshua D. HAIMSON, New York, NY (US)

(73) Assignee: Flatiron Health, Inc., New York, NY (US)

(21) Appl. No.: 16/971,608

(22) PCT Filed: Oct. 15, 2019
(86) PCT No.: PCT/US2019/056207

§ 371 Date: Aug. 20, 2020

Related U.S. Application Data
(60) Provisional application No. 62/747,428, filed on Oct. 18, 2018.

(51) Int. Cl. G16H 50/70; G06N 20/00; G06N 5/04; G16H 10/60; G16H 20/10; G06F 40/166; G06F 40/289

(52) U.S. Cl. CPC G16H 50/70; G06N 20/00; G06N 5/04; G16H 40/20; G16H 20/10; G06F 40/166; G06F 40/289; G16H 10/60

(57) ABSTRACT

A model-assisted selection system for predicting a date of an event relating to a patient may include at least one processor configured to obtain a medical record including a plurality of unstructured documents and obtain a model for predicting the date of the event. The at least one processor may further be configured to input the medical record into the model and assign, for each of the plurality of unstructured documents, a label from the model among a pre-event label, a mid-event label, a post-event label, and a non-event label. The at least one processor may also be configured to predict a start date of the event based on the labels of the plurality of unstructured documents and output the predicted start date.

### CROSS-REFERENCE TO RELATED APPLICATION

This application claims the benefit of priority of U.S. Provisional Application No. 62/747,428, filed on Oct. 18, 2018. The entire contents of the foregoing application are incorporated herein by reference in their entirety.

### BACKGROUND

### Technical Field

The present disclosure relates to a model-assisted system and method for predicting a date relating to an event.

### Background Information

It is important to understand the effectiveness of treatments (e.g., drugs administered orally) in real-world settings, particularly for diseases whose treatment landscapes are evolving rapidly. One such disease is renal cell carcinoma (RCC). Oral drugs are becoming increasingly common in oncology care. Since 2006, ten new targeted drugs have been approved for RCC, leading to uncertainties in guidelines that could benefit from studies using real-world evidence. In contrast to intravenous chemotherapy, which is administered in the clinic and carefully tracked via structured electronic health records (EHRs), oral drug treatments are typically self-administered and, therefore, less well-tracked. A challenge in conducting such studies on electronic health records (EHRs) is that treatment information often appears only in free text in unstructured clinic notes, a phenomenon particularly prevalent for oral cancer treatments, which are generally self-administered at home. Identifying and structuring this information is an important task in understanding a patient's treatment history. Additionally, most existing work on extracting drugs from EHRs has focused on discharge summaries. However, for chronic diseases such as cancer, drug treatment information is scattered longitudinally across clinic notes, requiring synthesis across the patient record.

Thus, there is a need for automated approaches for extracting drug treatment information from clinic notes.

### SUMMARY

Embodiments consistent with the present disclosure include systems and methods for predicting dates of an event associated with a patient. Embodiments of the present disclosure may overcome one or more aspects of existing techniques for predicting dates of an event by providing model-based, automated techniques for date prediction based unstructured data. For example, a trained model may receive a plurality of unstructured documents and label the unstructured documents. The model may also predict and output a start data of an event associated with a patient (e.g., taking a drug by the patient). The use of models in accordance with embodiments of the present disclosure thus allows for faster and more efficient prediction of dates of an event. In addition, the use of rules in accordance with embodiments of the present disclosure may be more accurate than extant techniques.

In one embodiment, a model-assisted selection system for predicting a date of an event relating to a patient may include at least one processor configured to obtain, from a storage device, a medical record of the patient. The medical record may include a plurality of unstructured documents. The at least one processor may also be configured to obtain a model for predicting the date of the event. The at least one processor may further be configured to input the medical record into the model and assign, for each of the plurality of unstructured documents, a label from the model. The label may be determined from among four labels, including a pre-event label, a mid-event label, a post-event label, and a non-event label. The pre-event label may indicate that a document relates to a date before the event. The mid-event label may indicate that a document relates to a date during the event. The post-event label may indicate that a document relates to a date after the event. The non-event label may indicate that a document is non-determinative or unrelated to the event. The at least one processor may also be configured to predict a start date of the event based on the labels of the plurality of unstructured documents and output the predicted start date.

In one embodiment, a model-assisted system for predicting a date of an event relating to a patient may include at least one processor configured to obtain a medical record of the patient. The medical record includes a plurality of unstructured documents. The at least one processor may further be configured to obtain a model for predicting the event. The at least one processor may also be configured to input the medical record into the model. According to the model and the medical record, for each of the plurality of unstructured documents, the at least one processor may further be configured to identify one or more time expressions in the each of the plurality of unstructured documents. The at least one processor may also be configured to determine one or more dates relating to the identified one or more time expressions. The at least one processor may further be configured to determine a probability score for the determined one or more dates for being associated with the beginning of the event, the ending of the event, or non-event date. The at least one processor may also be configured to predict a start date of the event based on the probability scores. The at least one processor may further be configured to output the predicted start date.

In one embodiment, a model-assisted system for predicting a date of an event relating to a patient may include at least one processor configured to obtain a first model for predicting the event. The at least one processor may also be configured to input a medical record of the patent into the first model. The medical record may include a plurality of unstructured documents. The at least one processor may further be configured to obtain, for each of the plurality of unstructured documents, a label from the first model. The label may be determined by the first model among four labels, including a pre-event label, a mid-event label, a post-event label, and a non-event label. The pre-event label may indicate that a document relates to a date before the event. The mid-event label may indicate that a document relates to a date during the event. The post-event label may indicate that a document relates to a date after the event. The non-event label may indicate that a document is non-determinative or unrelated to the event. The at least one processor may also be configured to predict a first preliminary start date of the event based on the labels of the plurality of unstructured documents. The at least one processor may further be configured to obtain, from the first model, a probability score for the first preliminary start date. The at least one processor may also be configured to obtain a second model for predicting the event. The at least one processor may further be configured to input the medical record into the second model. According to the second model and the medical record, for each of the plurality of unstructured documents, the at least one processor may also be configured to identify one or more time expressions in the each of the plurality of unstructured documents. The at least one processor may further be configured to determine one or more dates relating to the identified one or more time expressions and determine a probability score for the determined one or more dates for being associated with a beginning of the event, an ending of the event, or non-event date. The at least one processor may also be configured to predict a second preliminary start date of the event based on the determined probability scores. The at least one processor may further be configured to determine a probability score of the second preliminary start date. The at least one processor may also be configured to determine a start date of the event based on the first preliminary start date, the probability score of the first preliminary start date, the second preliminary start date, the probability score of the second preliminary start date.

Consistent with other disclosed embodiments, non-transitory computer-readable storage media may store program instructions, which are executed by at least one processing device and perform any of the methods described herein.

### BRIEF DESCRIPTION OF THE DRAWINGS

The accompanying drawings, which are incorporated in and constitute part of this specification, and together with the description, illustrate and serve to explain the principles of various exemplary embodiments. In the drawings:

FIG. 1A is a block diagram illustrating an exemplary system for predicting a date of an event associated with a patient, consistent with the present disclosure.

FIG. 1B is a block diagram illustrating an exemplary processing device for predicting a date of an event associated with a patient, consistent with the present disclosure.

FIG. 2 is a flowchart illustrating an exemplary medical record, consistent with the present disclosure.

FIG. 3 is a flowchart illustrating an exemplary process for training a model, consistent with the present disclosure.

FIG. 4 is a diagram illustrating an exemplary neural network, consistent with the present disclosure.

FIG. 5 is a flowchart illustrating an exemplary process for predicting a date of an event associated with a patient, consistent with the present disclosure.

FIG. 6 is a diagram illustrating an exemplary document timeline, consistent with the present disclosure.

FIG. 7 is a flowchart illustrating a flowchart illustrating an exemplary process for predicting a date of an event associated with a patient, consistent with the present disclosure.

FIG. 8A is a diagram illustrating exemplary mapped dates, consistent with the present disclosure.

FIG. 8B is a diagram illustrating exemplary revised sentences, consistent with the present disclosure.

FIG. 9 is a diagram illustrating exemplary document timelines, consistent with the present disclosure.

FIG. 10 is a flowchart illustrating a flowchart illustrating an exemplary process for predicting a date of an event associated with a patient, consistent with the present disclosure.

FIG. 11 is a flowchart illustrating a flowchart illustrating an exemplary process for predicting a date of an event associated with a patient, consistent with the present disclosure.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Embodiments herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, non-volatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

In this disclosure, a Temporally Integrated Framework for Treatment Intervals (TIFTI), a robust, generalizable framework for extracting oral drug treatment intervals from a patient's unstructured notes, is presented. TIFTI may leverage distinct sources of temporal information by breaking the problem down into a document-level sequence labeling task and a date extraction task.

According to one embodiment, a system may be configured to predict a start date of taking a drug by a patient. The system may input the name of the drug and a plurality of unstructured data, such as clinic visit notes into a model, which may predict whether the patient took the drug and if so, predict the time interval over which the patient took the drug. A user of the disclosed systems and methods may encompass any individual who may wish to access a patient's clinical experience and/or analyze patient data. Thus, throughout this disclosure, references to a “user” of the disclosed systems and methods may encompass any individual, such as a physician, a quality assurance department at a health care institution, and/or the patient.

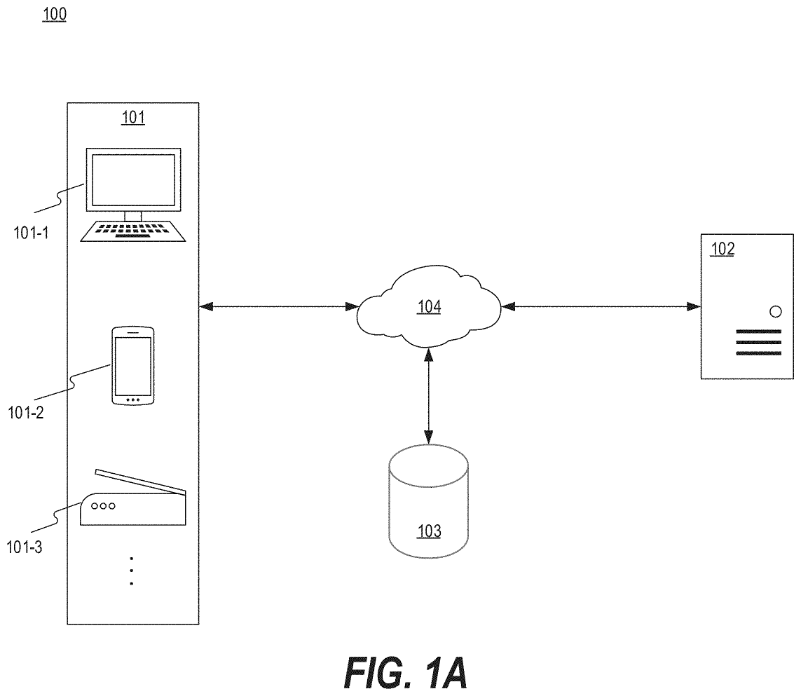

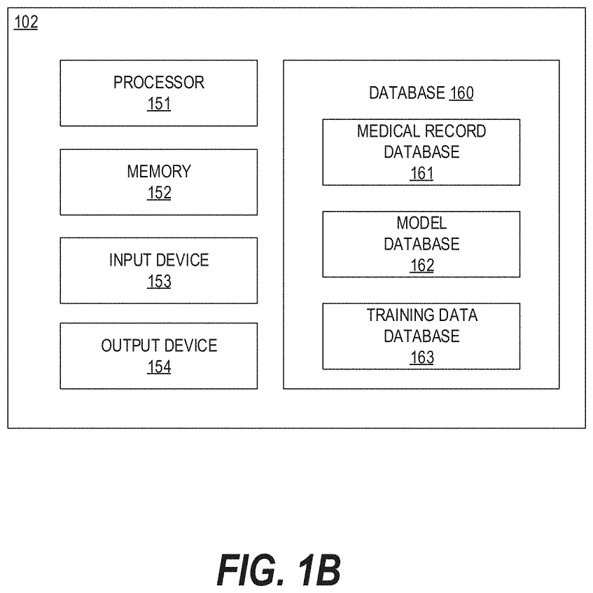

### FIGS. 1A-18 (Overview of the System)

FIG. 1A illustrates an exemplary system 100 for implementing embodiments consistent with the present disclosure, described in detail below. As shown in FIG. 1A, system 100 may include one or more data sources 101, a computing device 102, a database 103, and a network 104. It will be appreciated from this disclosure that the number and arrangement of these components is exemplary and provided for purposes of illustration. Other arrangements and numbers of components may be used without departing from the teachings and embodiments of the present disclosure.

One or more data sources 101 may obtain or generate a medical record (or medical data thereof) of a patient. For example, a data source may be a computer (e.g., computer 101-1 illustrated in FIG. 1A) in a clinic office configured to generate a medical record of a patient. A medical record may include medical data associated with the patient. The medical data may include structured data and/or unstructured data. Structured data may include quantifiable or classifiable data about the patient (e.g., as gender, age, race, weight). Unstructured data may include information about the patient that is not quantifiable or easily classified (e.g., a physician's notes or the patient's lab reports). Data sources 101 may further be configured to transmit the medical record (or medical data) to computing device 102 and/or database 103 via network 104.

Data sources 101 may include a computer (e.g., computer 101-1), a mobile device (e.g., smartphone 101-2), a scanner (e.g., scanner 101-3), a copier, a fax machine, a multi-function machine, a tablet computer, a personal digital assistant (PDA), or the like, or a combination thereof.

Computing device 102 may receive the medical record (or medical data) of the patient from one or more data sources 101 via network 104. In some embodiments, computing device 102 may receive medical data of the patient from one or more data sources 101 and compile the medical data into a medical record of the patient. Computing device 102 may also be configured to process the medical record (or medical data) to predict a date relating to an event associated with the patient. For example, computing device 102 may obtain a medical record of a patient and a model for predicting a start date of taking a particular drug by a patient (e.g., a trained neural network). Computing device 102 may further input the medical record into the model and obtain the prediction of the data from the model (e.g., via an output layer of the model). Computing device 102 may further output the prediction of the data to, for example, an output device. In some embodiments, computing device 102 may transmit the prediction to a physician or medical personnel associated with the patient. For example, computing device 102 may transmit the prediction to computer 101-1 located in a clinic office.

In some embodiments, computing device 102 may train a model for predicting a date relating to an event based on a training algorithm and training data. Alternatively or additionally, computing device 102 may obtain a model from a database (e.g., database 103 and/or database 160).

Database 103 may be configured to store information and data for one or more components of system 100. For example, database 103 may receive one or more medical records (or medical data thereof) from one or more data sources 101 and/or computing device 102 via, for example, network 104, and store the received data. Alternatively or additionally, database 103 may store one or more (untrained and/or trained) models and transmit one or more models to computing device 102 (e.g., if a request for a model is received) via network 104. In some embodiments, database 103 may store training data and transmit the training data to computing device 102 via, for example, network 104.

Network 104 may be configured to facilitate communications among the components of system 100. Network 104 may include a local area network (LAN), a wide area network (WAN), portions of the Internet, an Intranet, a cellular network, a short-ranged network (e.g., a Bluetooth™ based network), or the like, or a combination thereof.

FIG. 1B is a block diagram illustrating an exemplary computing device 102. Computing device 102 may include at least one processor (e.g., processor 151), a memory 152, an input device 153, an output device 154, and a database 160.

The processor may be configured to perform one or more functions described in this disclosure. The processor may comprise at least one processing device, such as one or more generic processors, e.g., a central processing unit (CPU), a graphics processing unit (GPU), or the like and/or one or more specialized processors, e.g., an application-specific integrated circuit (ASIC), a field-programmable gate array (FPGA), or the like.

The Computing device 102 may also include a memory 152 that may store instructions for various components of computing device 102. For example, memory 152 may store instructions that, when executed by processor 151, may be configured to cause processor 151 to perform one or more functions described herein.

Input device 153 may be configured to receive input from the user of computing device 102, and one or more components of computing device 102 may perform one or more functions in response to the input received. Output device 154 may be configured to output information and/or data to the user. For example, output device 154 may include a display configured to display a predicted date of an event to the user.

Database 160 may be configured to store various data and information for one or more components of computing device 102. For example, database 160 may include a medical record database 161 configured to store medical records of patients, from which processor 151 may receive one or more medical records. Database 160 may also include model database 162 configured to store one or more models for predicting a date of an event. A model may be a trained model or an untrained model. For example, processor 151 may receive a trained model for predicting a date of an event from model database 162. As another example, processor 151 may receive an untrained model and train the model based on training data (which may be stored in training data database 163). Database 160 may further include a training data database 163 configured to store training data, from which processor 151 may receive training data to train or modify a model.

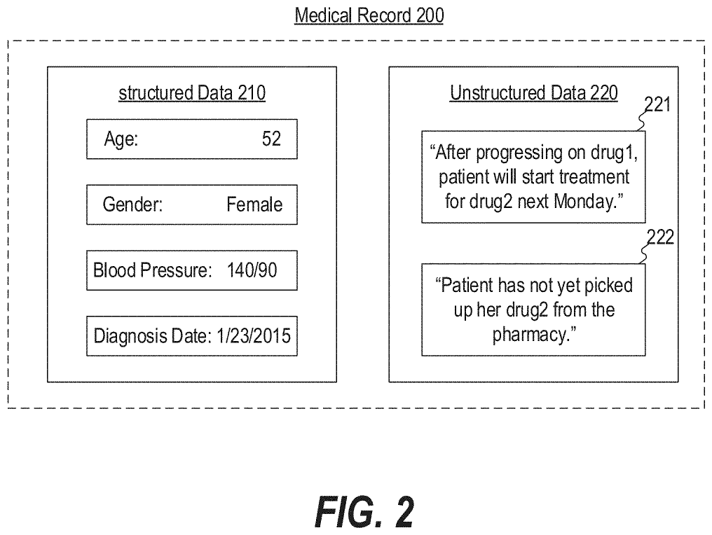

### FIG. 2 (Unstructured and Structured Data)

FIG. 2 illustrates an exemplary medical record 200 for a patient. Medical record 200 (or a portion thereof) may be received from data sources 101 and processed by computing device 102, as described above. Alternatively or additionally, medical record 200 may be stored in one or more databases (e.g., database 103, database 160). Computing device 102 may access and receive one or more medical records for further processing.

Medical record 200 may include both structured data 210 and unstructured data 220. Structured data 210 may include quantifiable or classifiable data about the patient, such as gender, age, race, weight, vital signs, lab results, date of diagnosis, diagnosis type, disease staging (e.g., billing codes), therapy timing, procedures performed, visit date, practice type, insurance carrier and start date, medication orders, medication administrations, or any other measurable data about the patient. Unstructured data 220 may include information about the patient that is not quantifiable or easily classified, such as physician's notes or the patient's lab reports. Unstructured data 220 may include information such as a physician's description of a treatment plan, notes describing what happened at a visit, descriptions of how a patient is doing, radiology reports, pathology reports, etc. In some embodiments, the unstructured data may be captured by an abstraction process, while the structured data may be entered by the health care professional or calculated using one or more algorithms. Unstructured data 220 may include a plurality of unstructured documents (e.g., exemplary unstructured documents 221 and 222 illustrated in FIG. 2).

In the data received from data sources 101, each patient may be represented by one or more records generated by one or more health care professionals or by the patient. For example, a doctor associated with the patient, a nurse associated with the patient, a physical therapist associated with the patient, or the like, may each generate a medical record (or a portion thereof) for the patient. In some embodiments, one or more records may be collated and/or stored in the same database. Alternatively or additionally, one or more records may be distributed across a plurality of databases. In some embodiments, the records may be stored and/or provided a plurality of electronic data representations. For example, the patient records may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process.

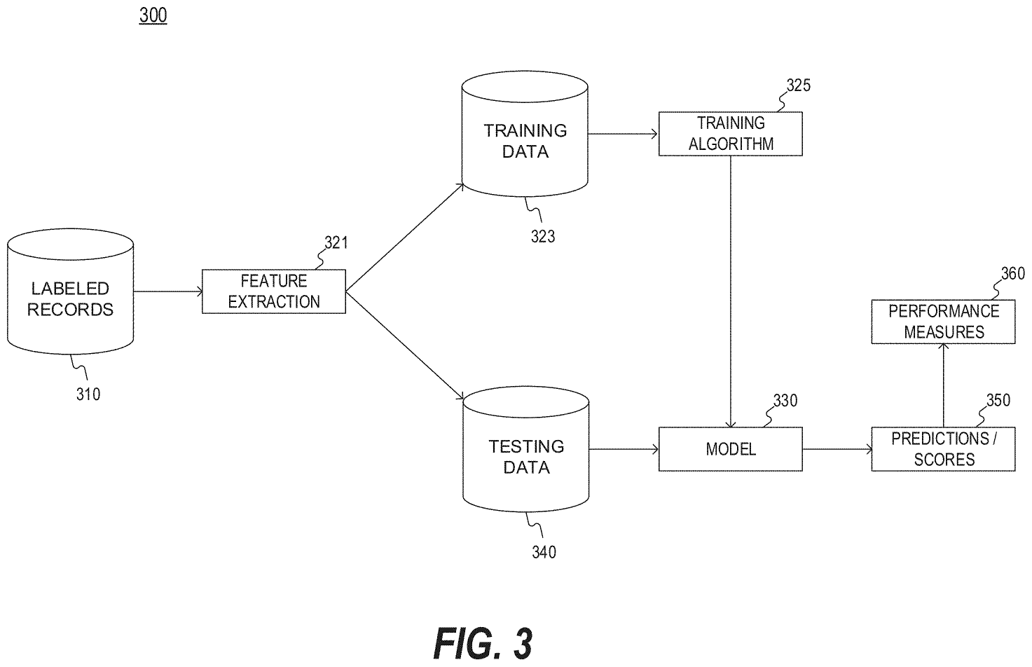

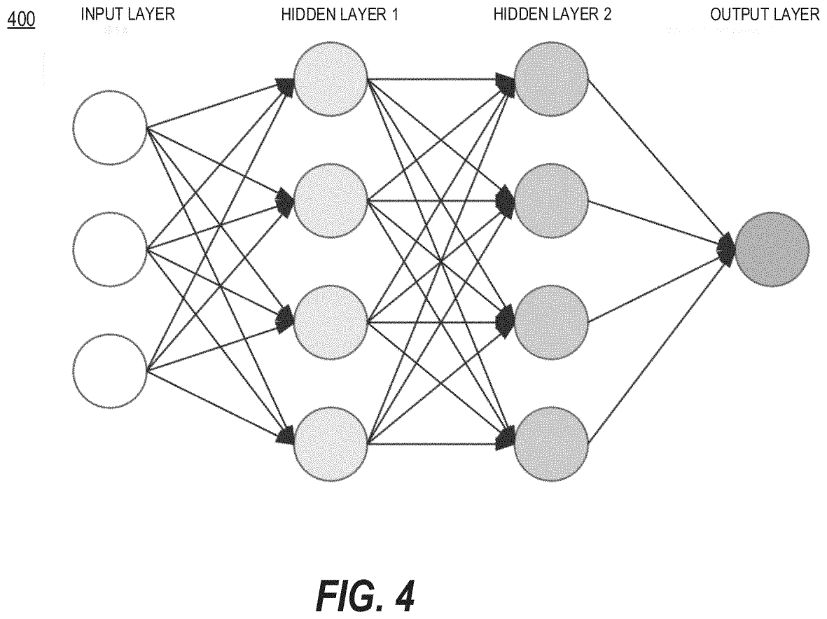

### FIGS. 3-4 (Training Process)

FIG. 3 illustrates an exemplary process 300 for training one or more models according to system 100 of FIG. 1A. Process 300 may be implemented to training one or more models described in this disclosure (e.g., a trained system, a neural network, etc.). For example, a model for labeling unstructured documents and determining a date of an event associated with a patient based on the labels may be trained based on process 300. As another example, a model for identifying one or more time expressions in unstructured documents and determining a date based on the identified time expressions may be trained based on process 300.

Labeled records 310 may be input to feature extraction 321. For example, labeled records 310 may be stored in one or more databases. Labeled records 310 may include data associated with a plurality of patients such that each patient is associated with one or more medical records. In some embodiments, a labeled record may include a plurality of unstructured documents (original or preprocessed) and a label associated with each of the unstructured documents. Alternatively or additionally, the labeled record may include a date and/or a period of an event (e.g., a start date, an end date, an time period, or the like, or a combination thereof). Alternatively or additionally, the labeled record may include one or more time expressions associated with an unstructured document and/or a revised unstructured document associated with the time expression(s) (as described elsewhere in this disclosure).

Feature extraction 321 may extract features (such as keywords, key phrases, or the like) from labeled records 310 and may score those features for a level of relevance to a date of the event. Accordingly, in some embodiments, the features may be represented as vectors.

A portion of the features extracted by feature extraction 321 may be collated with corresponding labels of records 310 and stored as training data 323. Training data 323 may then be used by one or more training algorithms 325. For example, training algorithm 325 may include logistic regression that may generate one or more functions (or rules) that relate extracted features to particular labels (e.g., a label assigned to a document, labeled date of the event, labeled period of the event, labeled time expression, labeled revised unstructured document), which may serve as ground truths. For example, training algorithm 325 may include simple l₂-regularized logistic regression, which may be featurized by ngrams. Additionally or alternatively, training algorithm 325 may include one or more neural networks that adjust weights of one or more nodes such that an input layer of features is run through one or more hidden layers and then through an output layer of labels (with associated probabilities). For example, the neural network may include an explicitly cascaded model, a long short-term memory (LSTM), or the like, or a combination thereof. Training algorithm 325 outputs one or more models 330.

FIG. 4 illustrates an exemplary neural network 400. Neural network 400 may include an input layer, one or more hidden layers, and an output layer. Each of the layers may include one or more nodes. In some embodiments, the output layer may include one node. Alternatively, the output layer may include a plurality of nodes, and each of the nodes may output a different data. The input layer may be configured to receive input (e.g., a medical record). In some embodiments, one or more hidden layers of the model may include at least one restraining module to implement the rules or restraints described in this disclosure.

In some embodiments, every node in one layer is connected to every other node in the next layer. A node may take the weighted sum of its inputs and pass the weighted sum through a non-linear activation function, the results of which may be output as the input of another node in the next layer. The training data may flow from left to right, and the final output may be calculated at the output layer based on the calculation of all the nodes.

Referring to FIG. 3, The other portion of the features extracted by feature extraction 321 may be collated with corresponding labels of records 310 and stored as testing data 340. Testing data 340 may be used to refine one or more models 330 to detect biases from under-inclusion or false positives from over-inclusion. The collated data 340 may then be placed through one or more models 330. One or more models 330 may produce predictions (or scores) 350 for testing data 340. Performance measures 360 may be used to refine one or more models 330, for example, by comparing predictions 350 to the labels of testing data 340. For example, as explained above, one or more models 330 may be re-trained (e.g., modified) to reduce deviations between the labels and predictions 350. The modifications may be based on one or more loss functions.

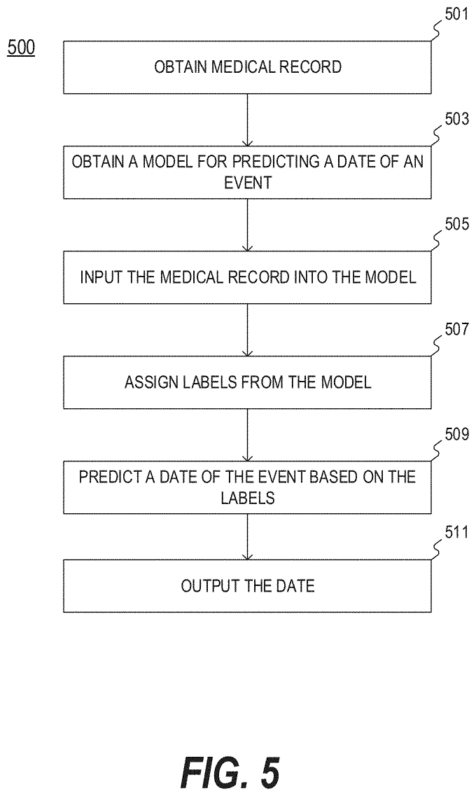

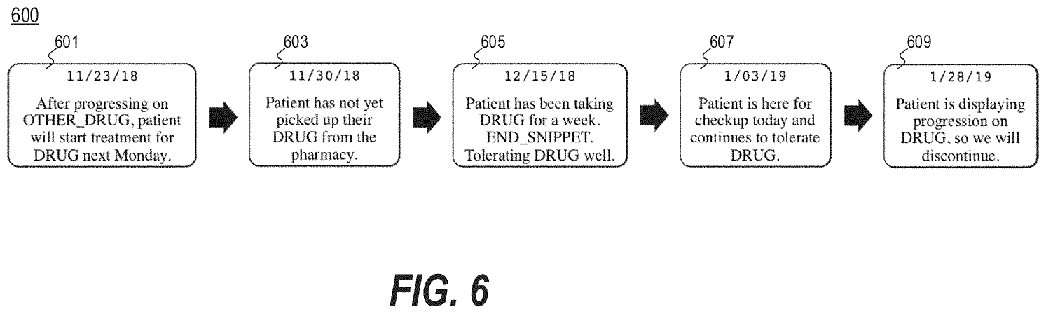

### FIGS. 5-6 (Document Timeline Sequence Labeling)

FIG. 5 is a flowchart of an exemplary process 500 for predicting one or more dates of an event associated with a patient, according to some embodiments described in this disclosure. While the descriptions of process 500 (and processes 700, 1000, and 1100 below) refer to the taking of a particular drug by a patient as an exemplary event, one having ordinary skill in the art would understand that an event is not limited to examples described in this disclosure. For example, an event may relate to a treatment received by a patient.

At step 501, computing device 102 may be configured to obtain a medical record of a patient from a storage device (e.g., database 103 and/or database 103). A medical record may include a plurality of unstructured documents. In some embodiments, the medical record may also include structured data, such as quantifiable or classifiable data about the patient. An unstructured document may include information about the patient that is not quantifiable or easily classified. Exemplary unstructured documents may include a patient's notes, clinic visit notes, physician's description of a treatment plan, lab reports, descriptions of how patient is doing, radiology reports, pathology reports, or the like, or a combination thereof. An unstructured document may be prepared by the patient, a nurse, a physician, a laboratory technician, or the like, or a combination thereof.

In some embodiments, computing device 102 may reprocess the received medical record. For example, for the unstructured document, computing device 102 may remove the document(s) and sentence(s) without a mention of the drug (either by the generic or brand name). Alternatively or additionally, computing device 102 may remove the redundancy of information included in the medical record. For example, computing device 102 may remove one or more sentences that appear in a document (e.g., a clinic note that occurred prior to the present note). Alternatively or additionally, computing device 102 may replace each mention of the drug with the placeholder “DRUG” and each mention of other commonly taken drugs with the placeholder “OTHER-DRUG.” This preprocess may ensure that the features learned by a model are generalizable across drugs.

Computing device 102 may also generate a preprocessed medical record. The preprocessed medical record may include a plurality of preprocessed unstructured documents based on the original unstructured documents. In some embodiments, two or more preprocessed unstructured documents may form a document timeline. A document timeline may include preprocessed unstructured documents sorted according to the time when a document was prepared, or a timestamp associated with the document.

FIG. 6 illustrates an exemplary document timeline 600. Document timeline 600 may include preprocessed unstructured documents 601, 603, 605, 607, and 609. Preprocessed unstructured documents 601, 603, 605, 607, and 609 may be obtained by computing device 102 by preprocessing unstructured documents (e.g., a plurality of clinic notes). For example, preprocessed unstructured document 601 may be generated by preprocessing a clinic note by a physician indicating that the patent will start treatment for a drug next Monday from the date of the note. During the preprocessing of the note, computing device 102 may replace the name of the non-target drug with placeholder “OTHER_DRUG” and the name of the target drug with placeholder “DRUG” to produce preprocessed unstructured document 601. In some embodiments, a preprocessing an unstructured document may include removing one or more sentences having no mention of the event or removing duplicate information, or the like, or a combination thereof.

In some embodiments, computing device 102 may input original unstructured documents into a model for preprocessing and obtain the preprocessed unstructured document from the model. In some embodiments, computing device 102 may input original unstructured documents into a model for preprocessing and predicting a date of an event (i.e., the model may be configured to preprocess the medical record and predict a date), and computing device 102 may receive the prediction from the model.

In some embodiments, the reprocessing may be part of step 701 of process 700, step 1001 of process 1000, and/or step 1101 of process 1100.

At step 503, computing device 102 may be configured to obtain a model for predicting the date of the event. In some embodiments, the model may include a trained model generated based on a training process (e.g., training process 300 as described elsewhere in this disclosure). In some embodiments, the model may be a simple l₂-regularized logistic regression, which may be featurized by ngrams. Alternatively or additionally, the model may include one or more neural networks. The neural network may include an explicitly cascaded model, a long short-term memory (LSTM), or the like, or a combination thereof.

In some embodiments, computing device 102 may obtain a model based on a particular event of interest. For example, computing device 102 may obtain a first model for a first drug, but may obtain a second model for a second drug. Alternatively or additionally, computing device 102 may obtain a model based on the demographic information relating to the patient of interest (e.g., age, gender).

In some embodiments, the model may include an input layer, one or more hidden layers, and an output layer. Each layer may include one or more nodes. The input layer may receive input (e.g., a drug name, a medical record, a preprocessed medical record, unstructured documents, preprocessed unstructured documents, or the like, or a combination thereof). In some embodiments, the output layer may include one node configured to output a data (e.g., a predicted start date of the event) or a set of data (a plurality of candidate dates and probabilities scores associated with the candidate dates). Alternatively, the output layer may include a plurality of nodes, and each of the nodes may output a different data. In some embodiments, every node in one layer is connected to every other node in the next layer. A node may take the weighted sum of its inputs and pass the weighted sum through a non-linear activation function, the results of which may be output as the input of another node in the next layer. The input data may flow through the layers, and the final output may be calculated at the output layer based on the calculation of all the nodes.

At step 505, computing device 102 may be configured to input the medical record into the model. For example, the user may select the medical record to be input to the model via input device 153. In some embodiments, the model may include an input layer, and computing device 102 may input the medical record into the input layer of the model. In some embodiments, the medical record may include at least one preprocessed unstructured document.

At step 507, computing device 102 may be configured to assign, for each of the plurality of unstructured documents, a label from the model. In some embodiments, the model may assign a label to an unstructured document based on the timestamp and/or time expression (explicitly or implicitly) indicated in the document. Alternatively or additionally, the model may take the timestamp and/or time expression indicated in another document (or multiple documents) into consideration in determining a label for an unstructured data. For example, the model may include a classification algorithm configured to assign a label to an unstructured document as the output from the output layer. By way of example, the model may assign to the unstructured document a label from, among four labels including a pre-event label (or referred herein as a “PRE” label), a mid-event label (or referred herein as a “MID” label), a post-event label (or referred herein as a “POST” label), and a non-event label (or referred herein as a “OTHER” label). The PRE label may indicate that a document relates to a date before the event. The MID label may indicate that a document relates to a date during the event. The POST label may indicate that a document relates to a date after the event. The OTHER label may indicate that a document is non-determinative or unrelated to the event.

In some embodiments, the model may implement rules or restraints to assign a label to an unstructured document. For example, the rules or restraints may be configured such that no document labeled MID may precede a PRE and no document labeled POST may precede a document labeled MID. In some embodiments, one or more hidden layers of the model may include at least one restraining module to implement the rules or restraints described in this disclosure.

In some embodiments, the model may include an output layer, and computing device 102 may be configured to assign, for each of the plurality of unstructured documents, a label from the output layer of the model

By way of example, referring to FIG. 6, the model may assign a PRE label to unstructured documents 601 and 603. The model may also assign a MID label to unstructured documents 605 and 607, and assign a POST label to unstructured document 609.

In some embodiments, the model may also determine a probability score for the assignment of the label to the unstructured document. Alternatively or additionally, the model may determine for each document a probability distribution across two or more labels. The model may also assign the label having the highest probability score as the label of the document.

At step 509, the model (or computing device 102) may be configured to predict a start date (or an end date, a period, or the like, or a combination thereof) of the event based on the labels of the plurality of unstructured documents.

In some embodiments, the model may implement rules or restraints to predict a date of the event. For example, one or more hidden layers of the model may include at least one restraining module to implement rules or restraints such that if there is no document labeled MID or POST, the model may output an indication that the drug was not taken. As another example, the rules may be implemented such that the start date may be set to the timestamp (or time expression) of the first document with a MID label (if exists) and the timestamp (or time expression) of the first document with a POST label (if exists). By way of example, referring to FIG. 6, the model may assign a MID label to unstructured document 605, which may be the first document with a MID label in document timeline 600. The model may also set Dec. 15, 2018, the timestamp of unstructured document 605, as the start date of taking the drug by the patient. Alternatively or additionally, the model may assign a POST label to unstructured document 609, which may be the first document with a POST label in document timeline 600. The model may set Jan. 28, 2019, the timestamp of unstructured document 609, as the end date of taking the drug by the patient. Alternatively or additionally, the model may determine a period of the event based on the start date and end date.

In some embodiments, the model may also determine a probability score for the predicted date(s). For example, the model may determine a probability score for the predicted start date of Dec. 15, 2018, and a probability score for the predicated end date of Jan. 28, 2019. The model may also output the dates and their corresponding probability scores. In some embodiments, the model may include an output layer, and the model may output the dates and their corresponding probability scores via the output layer.

In some embodiments, computing device 102 may receive the results of processing of the input by the model. For example, computing device 102 may receive the predicted date(s) and corresponding probability score(s) from the model. Alternatively or additionally, computing device 102 may receive from the model one or more labeled documents (e.g., one or more documents of documents 601, 603, 605, 607, and 609 with the assigned label(s)) and the probability scores associated with the labels.

At step 511, computing device 102 may be configured to output the predicted date(s). For example, computing device 102 may be configured to output the predicted start and end dates via output device 154 (e.g., a display). In some embodiments, computing device 102 may also be configured to output one or more results of the processing of the medical record by the model. For example, computing device 102 may be configured to output the probability scores associated with the dates.

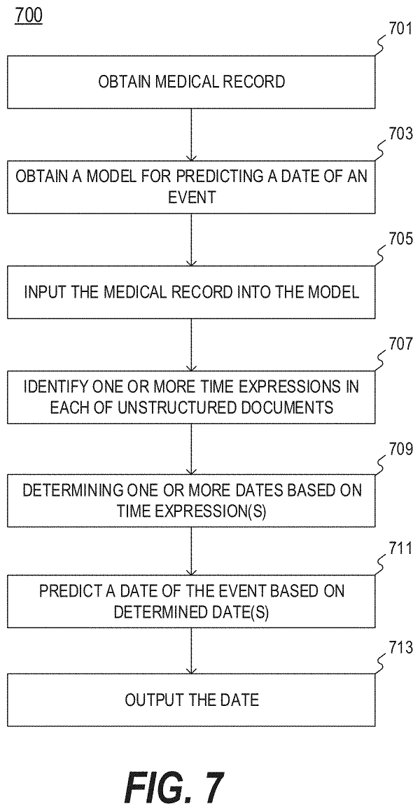

### FIGS. 7-9 (Time Expression Classification)

FIG. 7 is a flowchart of an exemplary process 700 for predicting one or more dates of an event associated with a patient, according to some embodiments described in this disclosure.

At step 701, computing device 102 may obtain a medical record of the patient. In some embodiments, computing device 102 may obtain a medical record based on one or more operations similar to those described in connection with step of 501 of process 500 as described elsewhere in this disclosure, and the detailed description is not repeated here for purposes of brevity.

At step 703, computing device 102 may obtain a model for predicting a date of an event associated with a patient. In some embodiments, computing device 102 may obtain a model based on one or more operations similar to those described in connection with step 503 of process 500 as described elsewhere in this disclosure, and the detailed description is not repeated here for purposes of brevity.

At step 705, computing device 102 may further be configured to input the medical record into the model. For example, the user may select the medical record to be input to the model via input device 153. In some embodiments, the medical record may include at least one preprocessed unstructured document. In some embodiments, the model may include an input layer, and computing device 102 may input the medical record into the input layer of the model.

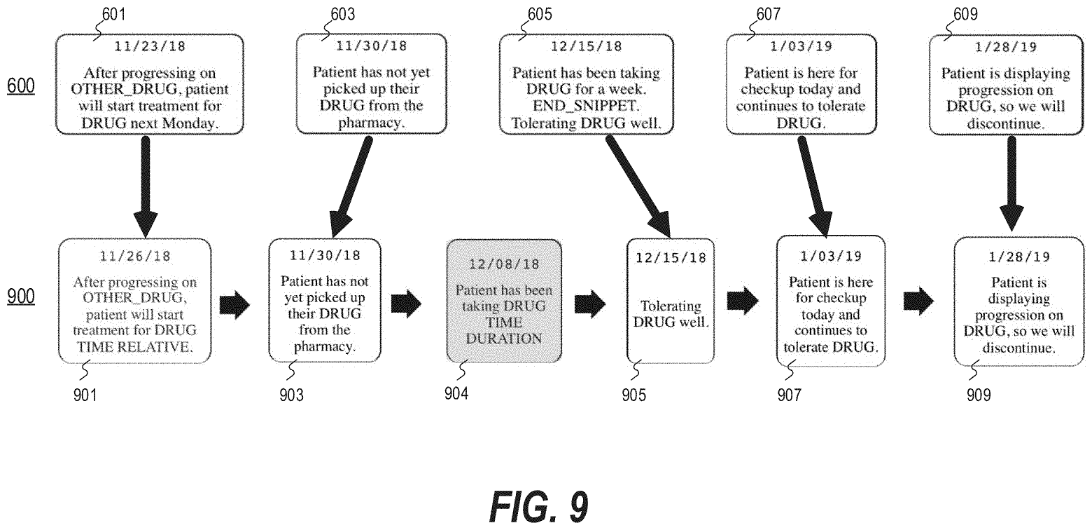

At step 707, according to the model and medical data, for each of the plurality of unstructured documents, computing device 102 may be configured to identify one or more time expressions in the each of the plurality of unstructured documents. A time expression may be a defined term (e.g., “Jan. 28, 2019”), a relative term (e.g., “next Monday”), a term referring to another date or event (e.g., “since the last visit”), or the like, or a combination thereof. By way of example, referring to FIG. 9, computing device 102 may input a medical record including a document timeline 600, which may include unstructured documents 601, 603, 605, 607, and 609. The model may be configured to identify one or more time expressions in the unstructured documents. The model may identify a time expression “next Monday” in unstructured document 601. The model may also identify the timestamp of the document as Nov. 23, 2018. As another example, the model may identify a time expression “for a week” in unstructured document 605. As a further example, the model may identify a time expression “today” in unstructured document 609

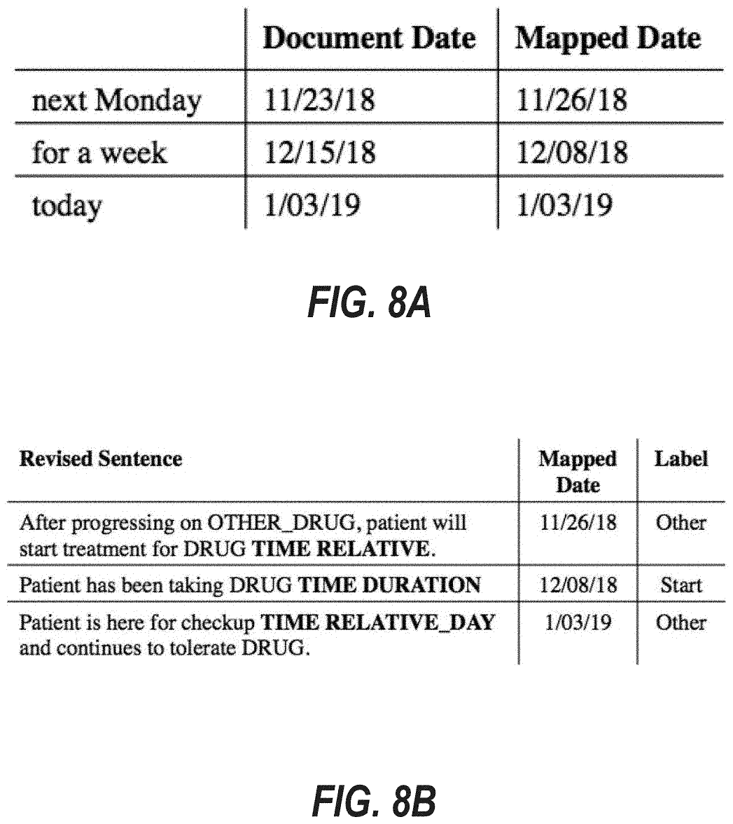

At step 709, the model may determine one or more dates relating to the identified one or more time expressions. By way of example, referring to FIG. 8A, for each of the time expressions, “next Monday,” “for a week,” and “today,” included in unstructured documents 601, 605, and 607 (illustrated in FIG. 9), respectively, the model may determine a date associated with the time expression (referred herein as a mapped date). In some embodiments, the model may use a regular expression-based temporal tagger that categorizes possible time expression types into one of a few buckets, such as specific dates (e.g. “November 27”) and relative dates (e.g. “last Tuesday”). The model may further determine a mapped date based on the identified date information.

In some embodiments, the model may determine a mapped date for a time expression based on the date of the document from which the time expression is identified. For example, as illustrated in FIG. 8A, the model may determine a mapped date of “Nov. 26, 2018” for the time expression “next Monday” identified in unstructured document 601 based on the document date of November 23 (which is a Friday). As another example, the model may determine a mapped date of “Dec. 8, 2018” for time expression “for a week” identified in unstructured document 605 based on the document date of Dec. 15, 2018. As a further example, the model may determine a mapped date of Jan. 3, 2019 for time expression “today” identified in unstructured document 607 based on the document date of Jan. 3, 2019.

In some embodiments, the model may determine a mapped date for a time expression based on the date of the document from which the time expression is identified and the date of another document. For example, a document may include a time expression referring to a previous clinic visit (e.g., “since the last visit till last Monday”). The model may identify the time expression “since the last visit” in this document and determine a mapped date (or a period) for the time expression based on the dates of this document and a document associated with the previous visit (i.e., the “last visit” referred in the document including the time expression).

In some embodiments, the model may be configured to revise the content of a document based on the identified time expression and its mapped date. By way of example, referring to FIG. 9, in the sentence “After progressing on OTHER_DRUG, patient will start treatment for DRUG next Monday” included in unstructured document 601, the time expression “next Monday” may be replaced with a time expression type name (referred to herein as “TIME BUCKET-NAME” such as “TIME RELATIVE,” “TIME DURATION,” or the like, or a combination thereof). For example, the time expression “next Monday” may be replaced with “TIME RELATIVE” as illustrated in FIG. 8B. As another example, time expression “today” included in unstructured document 607 may be replaced with “TIME RELATIVE_DAY.” In some embodiments, the model may generate a relationship between a mapped date and the term replacing the time expression associated with the mapped date (e.g., a lookup table similar to the table illustrated in FIG. 8B).

In some embodiments, the model may be configured to update the medical record received and generate the updated medical record including at least one document having revised or new content. By way of example, referring to FIG. 9, the model may update document timeline 600 and generate a simulated document timeline 900. The model may be configured to update document 601 by revising at least part of the content thereof (as described elsewhere in this disclosure) and generate an updated document 901. The model may also be configured to keep original documents 603, 607, and 609 as documents 903, 907, and 909. Alternatively, the model may update document 607 by replacing the time expression “today” with “TIME RELATIVE_DAY” (as illustrated in FIG. 8B). In some embodiments, the model may remove some information from a document. Alternatively or additionally, the model may generate a “pseudo” document based on one or more documents. By way of example, referring to FIG. 9, the model may be configured to update document 605 by removing the phrase “Patient has been taking DRUG for a week” from the document and generate document 905. The model may also generate a new “pseudo” document 904 based on the phrase removed from document 605 and the time expression identified in document 605. For example, the model may generate document 904 that includes the phrase “Patient has been taking DRUG TIME DURATION.” The model may further determine a mapped date of “Dec. 8, 2018” for the time expression “for a week” (and the type name “TIME DURATION”). The model may also set the mapped date as the date (or timestamp) of document 905.

In some embodiments, the model may also be configured to determine a probability score for the dates associated with the documents (e.g., a timestamp of a document, a date of a document, a mapped date associated with a document, or the like, or a combination thereof) for being associated with the beginning of the event (e.g., the start date), an ending of the event (e.g., the end date), or non-event date. By way of example, the model may determine a probability score for the mapped date of Dec. 8, 2018 (which is associated with document 904) for being associated with the beginning of taking the drug by the patient. Alternatively or additionally, the model may be configured to label document 904 (and/or the mapped date) as “Start,” as illustrated in FIG. 8B. As another example, the model may label the mapped dates of Nov. 26, 2018 and Jan. 3, 2019 (and/or the associated with documents) as “Other.”

In some embodiments, the model may determine whether to update a document based on the probability score for a date of the document for being associated with the beginning of the event (e.g., the start date), an ending of the event (e.g., the end date), or non-event date. For example, referring to FIG. 9, the model may determine whether the probability score for the date relating to document 605 for being associated with the beginning of the event is higher than a threshold (e.g., a number between 70%-99). If so, the model may not update document 605 (i.e., no creation of document 904 and/or no revision of document 905). If not, the model may proceed to update document 605 as described elsewhere in this disclosure.

At step 711, the model (or computing device 102) may be configured to predict one or more dates (and/or a period) associated with the event based on the probability scores associated with the dates of the documents. For example, the model may be configured to determine a date associated with a document (e.g., the timestamp of the document, the date of the document, a mapped date of the document) that has the highest probability score for being associated with the beginning (or the end) of taking the drug by the patient. By way of example, the model may determine that Dec. 8, 2019, which is associated with document 904, has the highest probability score for being associated with the beginning of the event, as the start date.

In some embodiments, the model (or computing device 102) may be configured to predict one or more dates (and/or a period) associated with the event based on dates associated with the documents and the probability scores for the dates for being associated with the beginning or ending of the event. For example, the model may be configured to determine an earliest document in the document timeline (e.g., a document having an earliest timestamp) that has a probability score for being associated with the beginning of the event higher than a threshold. As another example, the medical data may identify, among the plurality of the unstructured documents, one or more documents having a mid-event label, select, among the one or more documents having a mid-event label, a document having an earliest timestamp, and assign a date of the timestamp of the selected document as the starting date of the event.

At step 713, computing device 102 may be configured to output the predicted date(s). For example, computing device 102 may be configured to output the predicted start and end dates via output device 154 (e.g., a display). In some embodiments, computing device 102 may also be configured to output one or more results of the processing of the medical record by the model. For example, computing device 102 may also be configured to output the probability scores associated with the dates. As another example, computing device 102 may be configured to output the updated document timeline (e.g., updated document timeline 900). In some embodiments, the model may include an output layer configured to output one or more results of processing of the medical record by the model (e.g., one or more predicted dates, probability scores, updated documents, or the like, or a combination thereof).

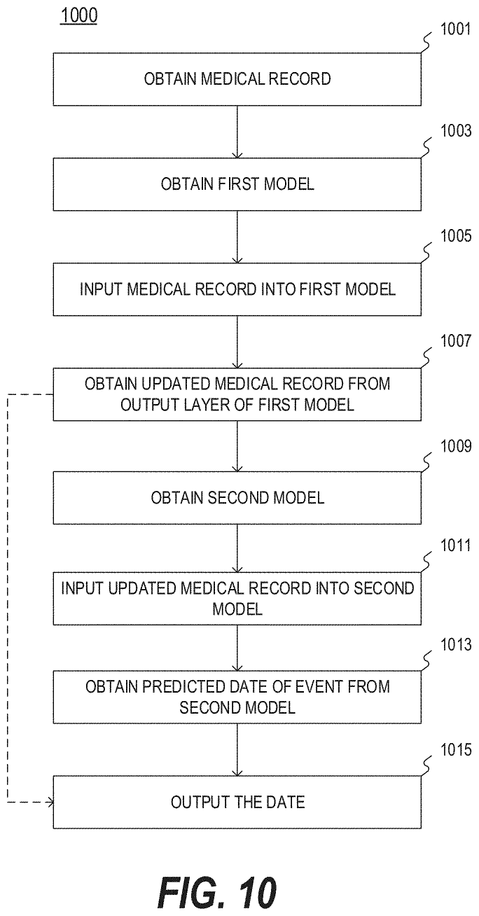

### FIG. 10 (Combination of Document Timeline Sequence Labeling and Time Expression Classification in Serial)

FIG. 10 is a flowchart of an exemplary process 1000 for predicting one or more dates of an event associated with a patient, according to some embodiments described in this disclosure. At step 1001, computing device 102 may obtain a medical record. In some embodiments, computing device 102 may obtain a medical record based on one or more operations similar to those described in connection with step 501 of process 500, and the detailed description is not repeated here from purposes of brevity.

At step 1003, computing device 102 may obtain a first model. In some embodiments, computing device 102 may obtain a first model similar to a model obtained in step 703 of process 700, and the detailed description is not repeated here from purposes of brevity.

At step 1005, computing device 102 may input the medical record into the first model. In some embodiments, computing device 102 may input the medical record into the first model based on one or more operations similar to those described in connection with step 705 of process 700 (or step 505 of process 500), and the detailed description is not repeated here from purposes of brevity.

At step 1007, the first model may generate and output an updated medical record, which may be received by computing device 102. The updated medical record may include at least one updated unstructured document having a mapped date. In some embodiments, the first model may generate one or more updated unstructured documents based on one or more operations similar to those described in connection with steps 707-711 of process 700. For example, the first model may be configured to identify one or more time expressions in an unstructured document of the medical record (similar to one or more operations described in connection with step 707 of process 700). The first model may also be configured to determine one or more dates (i.e., a mapped date) relating to the identified time expression(s) (similar to one or more operations described in connection with step 709 of process 700). The first model may further be configured to update the unstructured document by revising the content associated with the determined date relating to a time expression (similar to one or more operations described in connection with step 709 of process 700). In some embodiments, the first model may also be configured to create a “pseudo” document based on the determined date and the content of an original document. By way of example, the first model may generate document 904 illustrated in FIG. 9 and generate an updated document timeline 900.

In some embodiments, the first model may be configured to predict one or more preliminary dates (and/or a period) associated with the event based on the probability scores associated with the dates of the documents. For example, the first model may be configured to determine a date associated with a document (e.g., the timestamp of the document, the date of the document, a mapped date of the document) that has the highest probability score for being associated with the beginning (or the end) of taking the drug by the patient. The first model may also be configured to predict one or more preliminary dates (and/or a period) associated with the event based on dates associated with the documents and the probability scores for the dates for being associated with the beginning or ending of the event. The first model may further be configured to determine a probability score for the predicted preliminary date(s). If the probability score for the predicted preliminary date(s) is higher than a threshold (e.g., a number between 70%-99%), the preliminary date(s) may be set as the dates associated with the event (e.g., a start date, an end date), and process 1000 may proceed to step 1005, where computing device 102 may output the predicted date(s).

At step 1009, computing device 102 may obtain a second model. In some embodiments, computing device 102 may obtain a second model that is similar to the model obtained at step 503 of process 500, and the detailed description is not repeated here from purposes of brevity.

At step 1011, computing device 102 may input the updated medical record into the second model. By way of example, computing device 102 may input an updated medical record including updated document timeline 900 into the second model.

At step 1013, computing device 102 may obtain one or more predicated date associated with an event from the second model. In some embodiments, the second model may predict one or more dates associated with the event based on one or more operations similar to those described in connection with steps 507 and 509 of process 500, and the detailed description is not repeated here for purposes of brevity. By way of example, the second model may assign, for each of the updated documents (and/or original documents if they are not updated), a label based on the date associated with the updated document (e.g., a mapped date, a timestamp, a time expression, or the like, or a combination thereof). For example, the second model may assign a label, among PRE, MID, POST, and/or OTHER labels, to an updated (or original) document. The second model may further be configured to predict a start date (or an end date, a period, or the like, or a combination thereof) of the event based on the labels.

At step 1015, computing device 102 may output the predicted date(s) via, for example, output device 154. For example, computing device 102 may present the predicted start and end dates of taking the drug of the patient on a display. In some embodiments, computing device 102 may also present one or more results of the processing of the medical record and/or the updated medical record by the first and/or second models. By way of example, computing device 102 may present document timeline 500 and/or updated document timeline 900. As another example, computing device 102 may output the probability score of the predicted date(s).

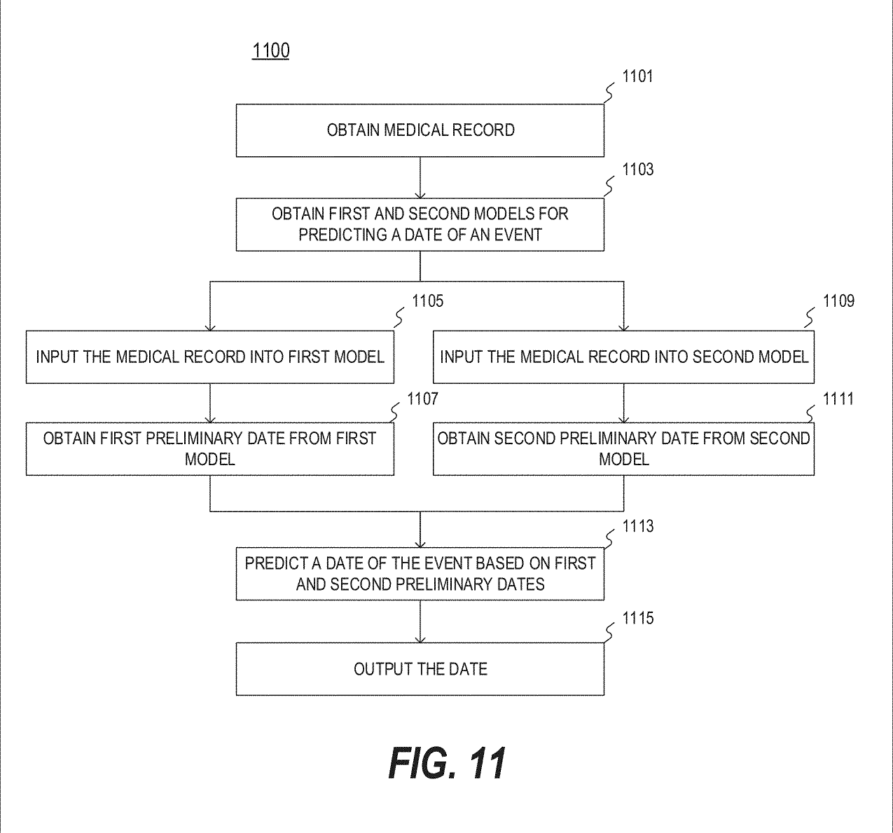

### FIG. 11 (Combination of Document Timeline Sequence Labeling and Time Expression Classification in Parallel)

FIG. 11 is a flowchart of an exemplary process 1100 for predicting one or more dates of an event associated with a patient, according to some embodiments described in this disclosure.

At 1101, computing device 102 may be configured to obtain a medical record. In some embodiments, computing device 102 may be configured to obtain a medical record based on one or more operations similar to those described in connection with step 501 of process 500 as described elsewhere in this disclosure, and the detailed description is not reheated here for purposes of brevity. By way of example, computing device 102 may obtain a medical record including a plurality of unstructured documents from a database. The unstructured documents may include preprocessed documents. Alternatively or additionally, the unstructured documents may include updated documents.

At 1103, computing device 102 may be configured to obtain a first model and a second model for predicting a date associated with an event. In some embodiments, the first model may include a model similar to the model obtained in process 700, and the second model may include a model similar to the model obtained in process 500. Detailed descriptions are not repeated here for purposes of brevity.

At 1105, computing device 102 may be configured to input the medical record into the first model. In some embodiments, computing device 102 may be configured to input the medical record into the first model based on one or more operations similar to those described in connection with step 705 of process 700 as described elsewhere in this disclosure, and detailed description is not repeated here for purposes of brevity.

At 1107, computing device 102 may be configured to obtain a first preliminary date associated with the event from the first model. The first preliminary date may include a start date and/or an end date of the event. In some embodiments, computing device 102 may be configured to predict the first preliminary date based on one or more operations similar to those described in connection with steps 707-711 of process 700 as described elsewhere in this disclosure, and the detailed description is not repeated here for purposes of brevity.

By way of example, the first model may be configured to identify one or more time expressions in an unstructured document of the medical record (similar to one or more operations described in connection with step 707 of process 700). The first model may also be configured to determine one or more dates (i.e., a mapped date) relating to the identified time expression(s) (similar to one or more operations described in connection with step 709 of process 700). The first model may further be configured to update the unstructured document by revising the content associated with the determined date relating to a time expression (similar to one or more operations described in connection with step 709 of process 700). The first model may also be configured to determine a probability score for a date associated with a document for being associated with the beginning of the event (e.g., the start date), an ending of the event (e.g., the end date), or non-event date. The first model (or computing device 102) may be configured to predict the first preliminary date (and/or a period) associated with the event based on dates associated with the documents and the probability scores for the dates for being associated with the beginning or ending of the event.

At 1109, computing device 102 may be configured to input the medical record into the second model. In some embodiments, computing device 102 may be configured to input the medical record into the second model based on one or more operations similar to those described in connection with step 505 of process 500 as described elsewhere in this disclosure, and the detailed description is not repeated here for purposes of brevity.

At 1111, computing device 102 may be configured to obtain a second preliminary date from the second model. The first preliminary date may include a start date and/or an end date of the event. In some embodiments, computing device 102 may be configured to obtain a second preliminary date from the second model based on one or more operations similar to those described in connection with steps 507 and 509 of process 500 as described elsewhere in this disclosure, and the detailed description is not repeated here for purposes of brevity. By way of example, the second model may be configured to assign a label to unstructured documents based on the timestamps and/or time expressions (explicitly or implicitly) indicated in the documents. Computing device 102 or the second model may also be configured to predict a second preliminary date (e.g., a start date or an end date) of the event based on the labels of the unstructured documents. In some embodiments, the model may also determine a probability score for the second preliminary date.

At 1113, computing device 102 may be configured to predict a date of the event based on the first and second preliminary dates. For example, the first preliminary date may include a first preliminary start date of taking the drug by the patient, and the second preliminary date may include a second preliminary start date. Computing device 102 may receive the first and second preliminary start dates and their corresponding probability scores from the first and second models. Computing device 102 may determine a start date based on the first and second preliminary dates. For example, computing device 102 may select one of the first and second preliminary dates that has a higher probability score as the date of the event. As another example, computing device 102 may determine a date between the first and second preliminary dates by, for example, selecting a date around the midpoint of the first and second preliminary dates, and assign this determined date as the date of the event.

At 1115, computing device 102 may be configured to output the date to the user. For example, computing device 102 may present the date to the user via output device 154 (e.g., a display).

### EXPERIMENTS AND RESULTS

### Examples

### Experimental Setup

Training data were obtained based on the clinic visit notes of a set of patients with metastatic RCC were obtained from a database, which is a longitudinal, demographically, and geographically diverse database derived from electronic health record (EHR) data. Oral drug regimens, along with their start and end dates, were extracted by clinical experts via chart review. These dates were used for labeling and held as ground truths. The units of observation were patient-drug pairs. Only pairs in which the clinic notes contained at least one mention of the drug (either by the generic or brand name) were considered. There were 8259 such patient-drug examples from 172 different practices. Of these, the drug was actually taken in 4410 (53%) examples; in the rest, the drug was mentioned in the notes but not taken.

80% of the labeled (or training) data were used for training models, and 20% were used for testing. The dataset was split such that no patients who appeared in the training set were in the test set.

The performance of the binary task of predicting whether the patient took the drug using the F₁ score. On true positive examples (those for which the model correctly predicted that the patient took the drug), the agreement of start and stop dates was measured as follows. Let Startᵢ(t) and Stopᵢ(t) be an indicator variable denoting whether for the i^{th} example, the predicted start or stop date matches the ground truth date within a window of t days. For example, Stopᵢ(7)=1 if either the patient is still taking the drug and the model correctly identifies this, or the last-taken date identified by the model is within a week of the ground truth. To measure overall date agreement, we used Startᵢ(t) and Stopᵢ(t), defined to be the mean over the true positives of the Startᵢ(t) and Stopᵢ(t) values.

To remain flexible and sensitive to dataset size, the TIFTI framework does not specify the classification algorithm for either sub-task. We tried multiple algorithms for each. On the document timeline sequence labeling task, we saw the best performance with a bidirectional LSTM over documents featurized by ngrams. On the time expression classification task, we saw the best performance with a simple l₂-regularized logistic regression, also featurized by ngrams. These optimizations, along with other hyperparameter tuning, were performed using 5-fold cross validation over the development set, optimizing on a combination of the F₁ score, Start (0), and Stop (0).

In order to perform well for rare drugs and generalize across diseases, TIFTI abstracts away the drug name during feature generation and models each drug independently. To test whether this design had the intended effect, we created a dataset of advanced non-small cell lung cancer (NSCLC) examples (a portion in the development set and a portion in the test set), using the same data preprocessing and feature generation process as for RCC. We then measured the performance on the NSCLC test set of the final TIFTI model trained on RCC and of a TIFTI model trained on NSCLC examples.

### Results

On the RCC test set, the model had an F₁ score of 0.944, a Start (0) score of 45.8%, a Stop (0) score of 52.4%, a Start (30) score of 85.9%, and a Stop (30) score of 77.6%. In an ablation study (Table 1), the two best performing models were the explicitly cascaded models. The model with the simulated document timeline slightly outperformed its counterpart with the original document timeline, both at 0 and 30 days, confirming that the pseudo-documents in the simulated timeline added useful context. This effect is only visible for the start date statistics, which is consistent with the fact that starts dates were more likely than stop dates to be explicitly mentioned in text.

TABLE 1
Ablation study of TIFTI framework, applied
to test set (about 1600 examples).

| | F₁ | Start | Stop | Start | Stop |
|---|---|---|---|---|---|
| Method | Score | (0) | (0) | (30) | (30) |
| Timeline Labeling | 0.943 | 23.8% | 51.4% | N/A | N/A |
| Simulated Timeline | 0.943 | 41.0% | 52.2% | N/A | N/A |
| Labeling | | | | | |
| Expression | 0.946 | 44.7% | 52.4% | 83.6% | 77.7% |
| Classification + Timeline | | | | | |
| Labeling | | | | | |
| Expression | 0.944 | 45.8% | 52.4% | 85.9% | 77.6% |
| Classification + Simulated | | | | | |
| Timeline Labeling | | | | | |
| (TIFTI) | | | | | |

On the NSCLC test set, the model trained on the RCC data had an F₁ score of 0.936, a Start (0) score of 49.1%, and a Stop (0) score of 57.1%. This performance was comparable to the performance on the RCC test set and was almost as high as the model trained on the NSCLC examples (F₁: 0.947, Start (0): 50.3%, Stop (0): 57.8%), indicating that the framework generalized as intended.

### CONCLUSION

TIFTI is a framework for extracting the spans of drug regimens from longitudinal clinic visit notes. TIFTI predicts the treatment interval over a simulated patient timeline formed by combining the temporal information from both free text and document timestamps. It predicted approximately 80% of dates within 30 days and generalized well to a new type of cancer.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer-readable media, such as secondary storage devices, for example, hard disks or CD ROM, or other forms of RAM or ROM, USB media, DVD, Blu-ray, 4K Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, Python, R, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A model-assisted system for predicting a date of an event relating to a patient, the system comprising:
at least one processor configured to:
obtain, from a storage device, a medical record of the patient, wherein the medical record includes a plurality of unstructured documents;
obtain a model for predicting the date of the event;
input the medical record into the model;
assign, for each of the plurality of unstructured documents, a label from the model, the label being determined from among four labels including a pre-event label, a mid-event label, a post-event label, and a non-event label, wherein:
the pre-event label indicates that a document relates to a date before the event;
the mid-event label indicates a document relates to a date during the event;
the post-event label indicates a document relates to a date after the event; and
the non-event label indicates a document is non-determinative or unrelated to the event;
predict a start date of the event based on the labels of the plurality of unstructured documents; and
output the predicted start date.

2. The system of claim 1, wherein the at least one processor is further configured to predict an end date of the event based on the labels of the plurality of unstructured documents.

3. The system of claim 1, wherein the at least one processor is further configured to:
determine, based on the labels of the plurality of unstructured documents, that no documents having a pre-event label, a mid-event label, or a post-event label have been identified; and
determine that no event occurred during a plurality of time periods associated with the plurality of unstructured documents.

4. The system of claim 1, wherein the event relates to a drug taken by the patient.

5. The system of claim 1, wherein the event relates to a treatment taken by the patient.

6. The system of claim 1, wherein the at least one processor is further configured to, for each of the plurality of unstructured documents, obtain, from the model, a probability score for each of the four labels.

7. The system of claim 1, wherein the at least one processor is further configured to, for each of the plurality of unstructured documents, determine, based on the model and one or more of the plurality of unstructured documents, a timestamp.

8. The system of claim 7, wherein predicting the start date of the event based on the labels of the plurality of unstructured documents comprises:
identifying, among the plurality of the unstructured documents, one or more documents having a mid-event label;
selecting, among the one or more documents having a mid-event label, a document having an earliest timestamp; and
assigning a date of the timestamp of the selected document as the starting date of the event.

9. The system of claim 7, wherein the at least one processor is further configured to:
identify, among the plurality of the unstructured documents, one or more documents having a post-event label;
select, among the one or more documents having a post-event label, a document having an earliest timestamp; and
assign a date of the timestamp of the selected document as an end date of the event.

10. The system of claim 1, wherein the at least one processor is further configured to perform a preprocessing for each of the plurality of unstructured documents, the preprocessing comprising at least one of: removing one or more sentences having no mention of the event or removing duplicate information.

11. The system of claim 1, wherein the model includes an input layer, one or more hidden layers, and an output layer.

12. A model-assisted system for predicting a date of an event relating to a patient, the system comprising:
at least one processor configured to:
obtain a medical record of the patient, wherein the medical record includes a plurality of unstructured documents;
obtain a model for predicting the event;
input the medical record into the model;
based on the model and the medical record, for each of the plurality of unstructured documents:
identify one or more time expressions in the each of the plurality of unstructured documents;
determine one or more dates relating to the identified one or more time expressions; and
determine a probability score for the determined one or more dates for being associated with a beginning of the event, an ending of the event, or non-event date;
predict a start date of the event based on the probability scores; and
output the predicted start date.

13. The system of claim 12, wherein the at least one processor is further configured to predict an end date of the event based on the probability scores.

14. The system of claim 12, wherein the event relates to a drug taken by the patient.

15. The system of claim 12, wherein the at least one processor is further configured to perform a preprocessing for each of the plurality of unstructured documents, the preprocessing comprising at least one of: removing one or more sentences having no mention of the event or removing duplicate information.

16. The system of claim 12, wherein for at least one of the plurality of unstructured documents, determining the one or more dates relating to the identified one or more time expressions comprises:
identifying a relative time expression in the at least one of the plurality of unstructured documents; and
determining a mapped date as the date for the at least one of the plurality of unstructured documents based on the identified relative time expression.

17. The system of claim 16, wherein determining the mapped date as the date for the at least one of the plurality of unstructured documents based on the identified relative time expression comprises:
determining the mapped date as the date for the at least one of the plurality of unstructured documents based on the identified relative time expression and another document of the medical record.

18. The system of claim 16, wherein at least one processor is further configured to obtain updated medical records from the model, the updated medical records including the revised at least one of the plurality of unstructured documents, the revised at least one of the plurality of unstructured documents including the mapped date that replaces the relative time expression.

19. The system of claim 18, wherein the at least one processor is further configured to:
process the updated medical record by:
obtaining a second model for predicting the event;
inputting the updated medical record into the second model;
for each of the documents of the medical record, obtaining a label from the second model, the label being determined by the second model among four labels including a pre-event label, a mid-event label, a post-event label, and a non-event label, wherein:
the pre-event label indicates that a document relates to a date before the event;
the mid-event label indicates a document relates to a date during the event;
the post-event label indicates a document relates to date after the event; and
the non-event label indicates a document is non-determinative or unrelated to the event; and
predicting a second start date of the event based on the labels of the documents of the updated medical record; and
outputting the predicted second start date.

20. A model-assisted system for predicting a date of an event relating to a patient, the system comprising:
at least one processor configured to:
obtain a first model for predicting the even;
input a medical record of the patent into the first model, wherein the medical record includes a plurality of unstructured documents;
obtain, for each of the plurality of unstructured documents, a label from the first model, the label being determined by the first model among four labels including a pre-event label, a mid-event label, a post-event label, and a non-event label, wherein:
the pre-event label indicates that a document relates to a date before the event;
the mid-event label indicates a document relates to a date during the event;
the post-event label indicates a document relates to a date after the event; and
the non-event label indicates a document is non-determinative or unrelated to the event;
predict a first preliminary start date of the event based on the labels of the plurality of unstructured documents;
obtain, from the first model, a probability score for the first preliminary start date;
obtain a second model for predicting the event;
input the medical record into the second model;
based on the second model and the medical record, for each of the plurality of unstructured documents:
identify one or more time expressions in the each of the plurality of unstructured documents;
determine one or more dates relating to the identified one or more time expressions; and
determine a probability score for the determined one or more dates for being associated with a beginning of the event, a ending of the event, or non-event date;
predict a second preliminary start date of the event based on the determined probability scores;
determine a probability score of the second preliminary start date; and
determine a start date of the event based on the first preliminary start date, the probability score of the first preliminary start date, the second preliminary start date, the probability score of the second preliminary start date.
